(19) United States
(12) Patent Application Publication
(10) Pub. No.: US 2026/0102504 A1
(43) Pub. Date: Apr. 16, 2026

(54) GLUCOSE-RESPONSIVE INSULIN COMPLEX, PREPARATION METHOD AND USE THEREOF

(71) Applicant: ZHEJIANG UNIVERSITY, Zhejiang (CN)

(72) Inventors: Zhen GU, Zhejiang (CN); Jinqiang WANG, Zhejiang (CN); Juan ZHANG, Zhejiang (CN)

(21) Appl. No.: 19/116,597

(22) PCT Filed: Sep. 26, 2023
(86) PCT No.: PCT/CN2023/121610

§ 371 Date: Mar. 28, 2025

(30) Foreign Application Priority Data
Sep. 29, 2022 (CN) 202211205085.8

(51) Int. Cl. A61K 47/64; A61P 3/10

(52) U.S. Cl. CPC A61K 47/645; A61P 3/10

(57) ABSTRACT

A glucose-responsive insulin complex, a preparation method and a use thereof. The glucose-responsive insulin complex includes phenylboronic acid-based polylysine and insulin with a diol structure, wherein interaction forces include: a dynamic electrostatic attraction, and a diol-PBA complexation force between the phenylboronic acid-based polylysine and the insulin with a diol structure.

### CROSS-REFERENCE TO RELATED APPLICATIONS

This application is a National Stage Application of PCT application No. PCT/CN2023/121610, filed on Sep. 26, 2023, which claims the priority and benefit of Chinese patent application No. 202211205085.8, filed on Sep. 29, 2022. The entireties of PCT application No. PCT/CN2023/121610 and Chinese patent application No. 202211205085.8 are hereby incorporated by reference herein and made a part of this specification.

### TECHNICAL FIELD

The disclosure relates to a glucose-responsive insulin complex, a preparation method and a use thereof.

### BACKGROUND ART

About 10% of diabetic patients worldwide need insulin treatment to maintain blood glucose levels within normal range. There are now a variety of insulin drugs on the market that can improve blood glucose control effect and reduce the frequency of injections. Under existing treatment methods, diabetics still need to inject insulin subcutaneously multiple times a day to coordinate with their daily diet and maintain stable fasting and postprandial blood glucose levels. This dosing regimen places a heavy physical and mental burden on diabetic patients, especially adolescents. In addition, the therapeutic window of insulin is narrow, so any improper insulin dosage may lead to hypoglycemia, especially nocturnal hypoglycemia, which leads to safety risk. Therefore, we need to design a new insulin delivery system to reduce the frequency of injections while improving the ability of insulin to regulate blood glucose.

Glucose-responsive insulin can mimic the function of human B cells, which releases insulin rapidly when blood glucose is high and releases slowly when blood glucose is low. Therefore, the glucose-responsive insulin can actively track blood glucose fluctuations. The insulin release rate can be dynamically adjusted to meet real-time dynamic demand for insulin. The therapeutic index of insulin is increased, so that the blood glucose control effect of insulin is improved. There are currently three major glucose-responsive mechanisms, including phenylboronic acid derivatives, glucose-binding molecules, and glucose oxidase. Phenylboronic acid-based glucose-responsive insulin formulation has been demonstrated to have rapid and robust in vitro and in vivo glucose-responsive insulin release properties, which are closer to clinical use. However, it is still difficult for current glucose-responsive insulin formulation to control blood glucose within a normal range for a long period after a single injection. How to achieve both rapid insulin release after meals and continuous and slow basal insulin release during fasting over a longer period after a single injection remains a considerable challenge for glucose-responsive insulin.

### SUMMARY

In order to solve the problem that the glucose-responsive insulin delivery system in the existing technology is difficult to control blood glucose within a normal range for a long period after a single injection, the present disclosure provides a glucose-responsive insulin complex, a preparation method and a use thereof. The glucose-responsive insulin complex of the present disclosure has beneficial glucose-responsive release performance. The glucose-responsive insulin complex can release insulin rapidly at high blood glucose concentrations. The glucose-responsive insulin complex can release insulin continuously and slowly to maintain basal blood glucose. Notably, the complex avoids the formation of subcutaneous fibrous capsules, which is crucial for the release of insulin from the complex reservoir.

In order to achieve the above object, the present disclosure adopts the following technical solutions.

The present disclosure provides a glucose-responsive insulin complex, which includes phenylboronic acid-based polylysine and insulin with a diol structure. In particular, interaction forces include: (1) a dynamic electrostatic attraction, and (2) a diol-PBA complexation force between the phenylboronic acid-based polylysine and the insulin with a diol structure.

In the present disclosure, the phenylboronic acid-based polylysine is obtained by grafting the polylysine with a carboxyl-modified phenylboronic acid compound. That is, in the phenylboronic acid-based polylysine, a phenylboronic acid group is grafted on a side chain of the polylysine by an amide bond.

In the present disclosure, the polylysine is at least one of an E-polylysine or an L-polylysine, preferably a phenylboronic acid-based L-polylysine.

In the present disclosure, a molecular weight of the polylysine is 2 k to 1000 k, preferably 30 k to 70 k.

In the present disclosure, the phenylboronic acid group can be a phenylboronic acid group substituted or unsubstituted with a substituent. In particular, the substituent is preferably one or more of a halogen and a nitro group. Preferably, the phenylboronic acid group is selected from one or more of following groups:

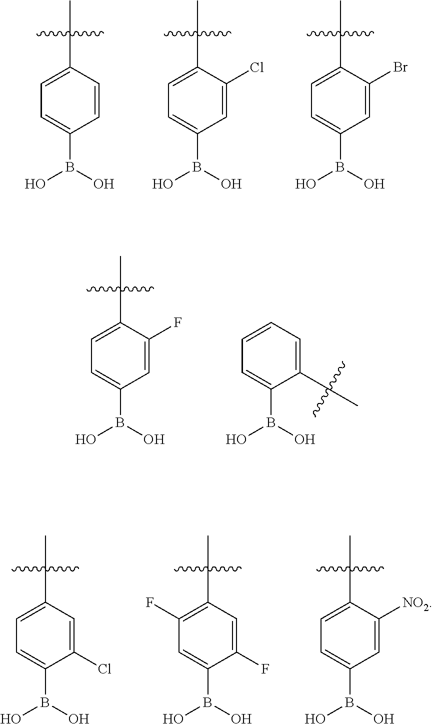

In the present disclosure, a phenylboronic acid grafting rate of the phenylboronic acid-based polylysine is preferably 25% to 75%, such as 30%, 35%, 40%, 45%, 50% or 60%. In particular, the phenylboronic acid grafting rate refers to the percentage of amino groups on polylysine covalently modified by phenylboronic acid groups to the total number of amino groups on polylysine before modification. The phenylboronic acid groups in the phenylboronic acid-based polylysine are randomly distributed.

In the present disclosure, preferably, the chemical structure of the phenylboronic acid-based polylysine can be shown by Formula I:

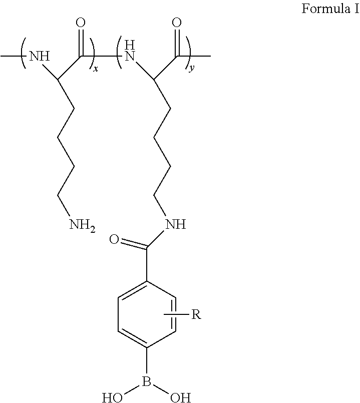

In particular, R represents a hydrogen atom or one or more substituents, and the substituents are as described above. The grafting rate of phenylboronic acid is y/(x+y).

In the present disclosure, the insulin with a diol structure can be prepared from insulin and a compound containing ortho- or meta-hydroxyl groups. In particular, the compound containing ortho- or meta-hydroxyl groups can be one or more of gluconic acid, dopamine, fructose, lactose, ribose, deoxyribose, β-D-pyranoside, trehalose and maltose, preferably gluconic acid, more preferably D-gluconic acid. When the compound containing ortho- or meta-hydroxyl groups is gluconic acid, the insulin with a diol structure is called gluconoyl insulin (Glu-insulin).

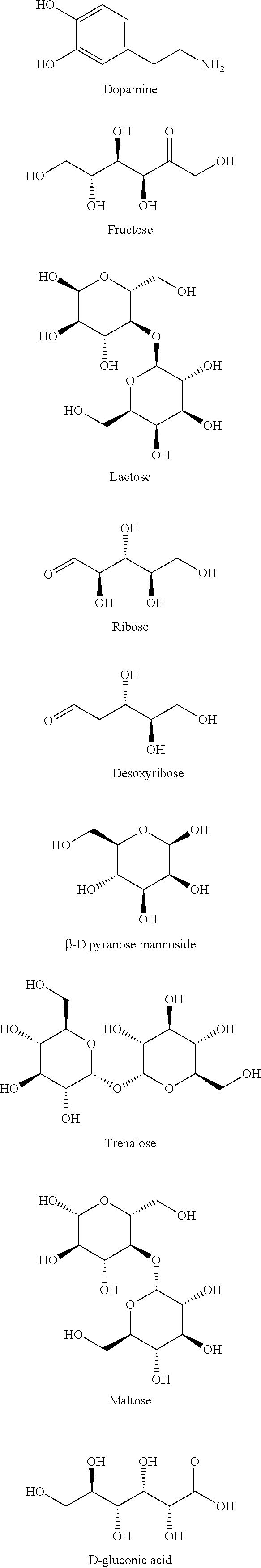

In the present disclosure, the insulin loading rate in the glucose-responsive insulin complex can be 5%-80%, preferably 40%-60%, more preferably 40%-50%, and further more preferably 45%-48%. In particular, the percentage is a mass percentage of the insulin with a diol structure in the glucose-responsive insulin complex to the glucose-responsive insulin complex.

In the present disclosure, the representative chemical structure of the glucose-responsive insulin complex is shown in Formula II:

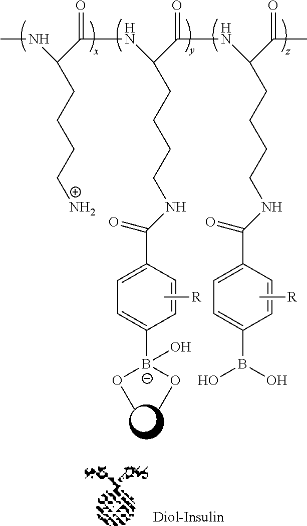

In particular, R represents a hydrogen atom or one or more substituents, and the substituents are as described above. The grafting rate of phenylboronic acid is y+z/(x+y+z).

The disclosure provides a method for preparing the above-mentioned glucose-responsive insulin complex, including the following steps: mixing an aqueous solution of the phenylboronic acid-based polylysine with an aqueous solution of the insulin with a diol structure, adjusting the pH to the range of 6.5 to 8.0.

In the present disclosure, a mass ratio of the insulin with a diol structure to the phenylboronic acid-based polylysine can be 1:(0.5-10), preferably 1:(1-2), such as 1:1.5 or 1:1.

In the present disclosure, a concentration of the aqueous solution of the phenylboronic acid-based polylysine can be 1 to 200 mg/mL, such as 10 mg/mL.

In the present disclosure, a preparation method of the aqueous solution of the phenylboronic acid-based polylysine can be: dissolving the phenylboronic acid-based polylysine in weakly acidic water. In particular, the pH of the weakly acidic water can be 2.0 to 7.0, preferably 2.0 to 3.0, the weakly acidic water can be phosphate buffer, deionized water or pure water.

In the present disclosure, the phenylboronic acid-based polylysine can be prepared by conventional methods in the art.

In certain preferred implementations, the method for preparing phenylboronic acid-modified polylysine includes the following steps: mixing a polylysine solution and a solution of a carboxyl-modified phenylboronic acid compound and then performing a grafting reaction.

In particular, the molar ratio of the structural unit of polylysine to the carboxyl-modified phenylboronic acid compound is preferably 4:(3-1).

In the present disclosure, the solvent in the polylysine solution can be a conventional aqueous solvent in the art that can dissolve polylysine, preferably deionized water or pure water.

In the present disclosure, a concentration of the solution of the polylysine is preferably 1 to 200 mg/mL, such as 10 mg/mL.

In the present disclosure, the solvent in the solution of the carboxyl-modified phenylboronic acid compound can be a conventional water-miscible organic solvent in the art that can dissolve the carboxyl-modified phenylboronic acid compound, preferably dimethyl sulfoxide (DMSO) or N, N-dimethylformamide (DMF).

In the present disclosure, the carboxyl-modified phenylboronic acid compound may be p-carboxylphenylboronic acid or o-carboxylphenylboronic acid substituted or unsubstituted with a substituent. In particular, the substituent may be one or more of a halogen and a nitro group. Preferably, the carboxyl-modified phenylboronic acid compound is selected from one or more of the following compounds:

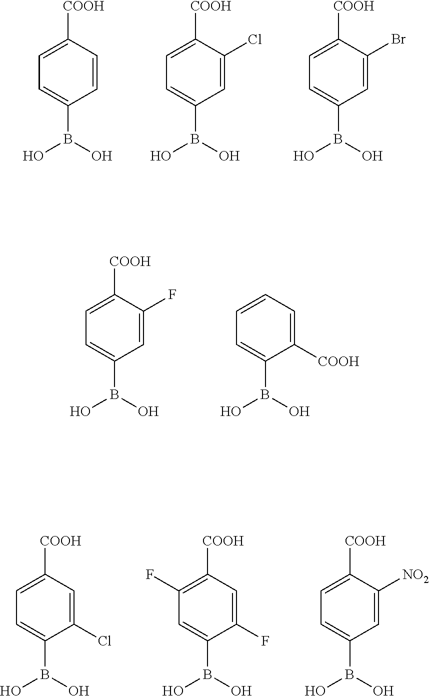

More preferably, the carboxyl-modified phenylboronic acid compound is 4-carboxy-3-fluorophenylboronic acid.

In the present disclosure, a concentration of the solution of the carboxyl-modified phenylboronic acid compound is preferably 1 to 500 mg/mL, such as 24 mg/mL.

In the present disclosure, preferably, the mixing method includes dropping the solution of the carboxyl-modified phenylboronic acid compound into the polylysine solution and then stirring. In particular, the stirring time is preferably 5 min to 24 h, such as 30 min.

In the present disclosure, preferably, the grafting reaction further includes a dialysis step. In particular, the dialysis can be performed in deionized water using a conventional dialysis bag in the art. The molecular weight cut-off of the dialysis bag is preferably 1 k to 10 k. The purpose of the dialysis is to remove free carboxyl-modified phenylboronic acid compounds.

In the present disclosure, preferably, the grafting reaction further includes a freeze-drying step. When a dialysis step is further included after the grafting reaction, the freeze-drying is performed after the dialysis. A white solid is obtained after freeze-drying.

In the present disclosure, a concentration of the aqueous solution of the insulin with a diol structure can be 1 to 200 mg/mL, preferably 1 to 100 mg/mL, such as 10 mg/mL.

In the present disclosure, a preparation method of the aqueous solution of the insulin with a diol structure can be: dissolving the insulin with a diol structure in weakly acidic water. In particular, the pH of the weakly acidic water is 2.0 to 7.0, preferably 2.0 to 3.0. The weakly acidic water can be phosphate buffer, deionized water or pure water.

In the present disclosure, the insulin with a diol structure is preferably Glu-insulin. In particular, the Glu-insulin can be prepared by conventional methods in the art.

Specifically, the preparation method of the Glu-insulin includes: (1) reacting gluconic acid with N-hydroxysuccinimide or TSTU (2-succinimidyl-1,1,3,3-tetramethyluronium tetrafluoroborate) in a solvent at first; (2) subsequently adding the reactants to an insulin aqueous solution for reaction, adjusting the pH to the range of 7 to 8, and post-treating the solution to obtain the insulin.

In step (1), the solvent can be any conventional solvent in the art, preferably DMSO.

In step (1), the reaction is preferably carried out at room temperature.

In step (1), the molar ratio of the gluconic acid to the N-hydroxysuccinimide or TSTU is preferably 1:(1-1.2).

In step (2), the insulin is preferably recombinant human insulin.

In step (2), the reaction is preferably carried out under ice bath conditions.

In step (2), the concentration of the insulin aqueous solution is preferably 0.2 to 100 mg/mL.

In step (2), the pH of the insulin aqueous solution is preferably 7 to 8.

In step (2), the post-treatment preferably includes dialysis and ion exchange column separation.

In particular, the dialysis can be performed in deionized water using a conventional dialysis bag in the art, and the molecular weight cut-off of the dialysis bag is preferably 1 k to 3.5 k.

In particular, the ion exchange column is preferably an anion exchange column.

In the present disclosure, preferably after mixing an aqueous solution of the phenylboronic acid-based polylysine with an aqueous solution of the insulin with a diol structure, adjusting the pH to the range of 6.5 to 7.4.

In the present disclosure, the white flocculent precipitate formed after adjusting the pH is the glucose-responsive insulin complex. The glucose-responsive insulin complex is soluble or easily soluble in weakly acidic water, and insoluble or slightly soluble in weakly alkaline water.

In the present disclosure, a step of centrifugation can be further included after adjusting the pH. The purpose of the centrifugation is to collect the white flocculent precipitate and remove the free insulin with a diol structure in the supernatant. The concentration of free insulin in the supernatant obtained by the centrifugation measured by Bradford assay reagent is no more than 10%, preferably no more than 5%.

In the present disclosure, an insulin encapsulation rate of the method is above 90%, preferably above 95%. The insulin encapsulation rate refers to the percentage of the insulin with a diol structure complexed with the phenylboronic acid-based polylysine (complexed amount) to the total mass of the insulin with a diol structure in the aqueous solution of the insulin with a diol structure (initial feed amount).

The glucose-responsive insulin complex of the disclosure is an amorphous flocculent precipitate, which can form an insulin reservoir after subcutaneous injection. Under the condition of high blood glucose concentrations, the combination of glucose with high concentrations and phenylboronic acid groups significantly reduces the positive charge density of the phenylboronic acid-based polylysine portion. Besides, the phenylboronic acid ester bond is broken, so that the interaction between the insulin with a diol structure and the polymer is decreased. It promotes the release of insulin, so that insulin has a beneficial release performance in response to high-concentration glucose. The restoration of the blood glucose to a normal level is promoted. When blood glucose returns to normal levels, only a small amount of insulin is in a free state. The occurrence of hypoglycemia is reduced.

The disclosure also provides a use of the above-mentioned glucose-responsive insulin complex in preparing an insulin formulation for treating diabetes.

In the present disclosure, the diabetes may be type 1 diabetes or type 2 diabetes that need insulin treatments.

In the present disclosure, the glucose-responsive insulin complex is resuspended in phosphate buffer or physiological saline and administered by subcutaneous injection.

The disclosure also provides a method for treating diabetes in patients. The method includes the step of administering the glucose-responsive insulin complex to the patients.

The disclosure also provides a drug for treating diabetic patients. The drug includes the glucose-responsive insulin complex.

The disclosure also provides the glucose-responsive insulin complex for use in treating diabetes.

On the basis of being in accordance with the common sense in the art, the above-mentioned preferred conditions can be arbitrarily combined to obtain the preferred instances of the present disclosure.

The reagents and raw materials used in the present disclosure are commercially available.

The positive progressive effect of the disclosure lies in:

The glucose-responsive insulin complex of the present disclosure has a good glucose-responsive release performance. The insulin can be released rapidly at high blood glucose concentrations. The insulin can be released continuously and slowly (long-term release) at normal blood glucose concentrations. The glucose-responsive insulin complex of the disclosure is an amorphous flocculent precipitate and can form an insulin reservoir after subcutaneous injection. Under the condition of high blood glucose concentrations, the combination of glucose with high concentrations and phenylboronic acid groups significantly reduces the positive charge density of the phenylboronic acid-based polylysine portion. Besides, the phenylboronic acid ester bond is broken, so that the interaction between the insulin with a diol structure and the polymer is decreased. It promotes the release of insulin, so that the insulin has a beneficial release performance in response to high-concentration glucose. The restoration of the blood glucose to a normal level is promoted. When blood glucose returns to normal levels, only a small amount of insulin is in a free state. The occurrence of hypoglycemia is reduced. Notably, the insulin complex does not cause strong inflammatory responses under the skin. The formation of fibrous capsules can be avoided, which creates favorable conditions for the release of insulin from the glucose-responsive insulin complex.

The glucose-responsive insulin complex of the present disclosure can form an insulin reservoir after subcutaneous injection. Sustained insulin release maintains blood glucose within the normal range for up to one week. A high dose of insulin can be administered at one time and the compliance of diabetic patients is improved.

### BRIEF DESCRIPTION OF THE DRAWINGS

FIG. 1 is the schematic diagram of the mechanism of glucose-responsive insulin complex formation and insulin release.

FIG. 2 is the MALDI-TOF mass spectrum of synthetic Glu-insulin.

FIG. 3 is the MS/MS spectrum of Glu-insulin.

FIG. 4 is the 1H-NMR spectrum of the synthesized 4-carboxy-3-fluorobenzeneboronic acid (FPBA)-modified poly-L-lysine (PLL-FPBA) in D2O.

FIG. 5 is the MALDI-TOF mass spectrum of PLL-FPBA.

FIG. 6 is the 11B-NMR spectrum of PLL-FPBA.

FIG. 7 is the standard curve established for calculating Glu-insulin concentration.

FIG. 8 is the encapsulation rate of insulin at various feed ratios.

FIG. 9 is the characterization of AKT signal phosphorylation stimulated by recombinant human insulin or Glu-insulin.

FIG. 10 is the representative fluorescence image of the glucose-responsive insulin complex.

FIG. 11 is the scanning electron microscopy (SEM) and cryo-transmission electron microscopy (cryo-TEM) image of the glucose-responsive insulin complex.

FIG. 12 is the glucose-responsive insulin release diagram of the glucose-responsive insulin complex in vitro.

FIG. 13 is the pulsatile insulin release profile from the glucose-responsive insulin complex (the insulin complex was alternately exposed to 100 and 400 mg/dL glucose solutions).

FIG. 14 is the blood glucose level in type 1 diabetic mice treated with Glu-insulin and recombinant human insulin.

FIG. 15 is the blood glucose level in type 1 diabetic mice treated with subcutaneous injection of the glucose-responsive insulin complex (20 mg/kg).

FIG. 16 is the blood glucose level in type 1 diabetic mice treated with the commercial long-acting insulin preparation insulin glargine.

FIG. 17 is the blood glucose level in type 1 diabetic mice during intraperitoneal glucose tolerance test after treatment with the glucose-responsive insulin complex.

FIG. 18 is the blood glucose level in type 1 diabetic mice during intraperitoneal glucose tolerance test after treatment with the insulin glargine.

FIG. 19 is the blood glucose level and plasma insulin level in type 1 diabetic mice during intraperitoneal glucose tolerance test after treatment with the glucose-responsive insulin complex.

FIG. 20 is the blood glucose level in type 1 diabetic mice injected multiple times subcutaneously with the glucose-responsive insulin complex (black arrows indicate three injections of the complex).

FIG. 21 is the blood glucose level in type 1 diabetic minipigs with various treatments.

FIG. 22 is the level of major serum biochemical indices in type 1 diabetic mice after subcutaneous injection of the glucose-responsive insulin complex.

FIG. 23 is the representative fluorescence image of PLL-FPBA in the skin and major organs after subcutaneous injection of the glucose-responsive insulin complex.

FIG. 24 is the representative image of H&E and Masson's trichrome staining of the skin at the site of the insulin complex injection.

FIG. 25 is the representative H&E and Masson's trichrome staining image of the skin 2 weeks after subcutaneous implantation.

FIG. 26 is the representative H&E and Masson's trichrome staining image of the skin 4 weeks after subcutaneous implantation.

FIG. 27 is the representative H&E and Masson's trichrome staining image of the skin 12 weeks after subcutaneous implantation.

FIG. 28 is the immunofluorescence (macrophage biomarker, F4/80, in red; α-smooth muscle actin, α-SMA, in green; cell nuclei in blue) and immunohistochemistry (TNF-α, IL-6, IL-10, IL-12, IL-17) staining of the skin at the implantation site 2 weeks after subcutaneous implantation.

FIG. 29 is the integrated optical density statistics of immunohistochemistry (TNF-α, IL-6, IL-10, IL-12, IL-17) of the skin at the implantation site 2 weeks after subcutaneous implantation.

### DETAILED DESCRIPTION

The present disclosure is further described below by way of examples, but the present disclosure is not limited to the scope of the examples. The experimental methods in the following examples without specifying specific conditions were carried out according to conventional methods and conditions, or selected according to the product instructions.

The manufacturers or models of the reagents or instruments used in the following examples are shown in Table 1.

TABLE 1

| Reagents or Instruments | Model | Manufacturer |
|---|---|---|
| 4-carboxy-3- | CAS: 120153-08-4 | Aladdin |
| fluorophenylboronic acid | | |
| Gluconic acid | CAS: 526-95-4 | Sigma-Aldrich |
| Benzoic acid | CAS: 65-85-0 | Aladdin |
| Polycaprolactone | CAS: 24980-41-4 | Aladdin |
| Silicone | Sylgard 184 | DOWSIL |
| N-Hydroxysuccinimide | CAS: 6066-82-6 | Aladdin |
| Poly-L-lysine | CAS: 25988-63-0 | Sigma-Aldrich |
| Recombinant human insulin | No. A113811IJ | Thermo Fisher |
| Streptozotocin | CAS: 18883-66-4 | Sigma-Aldrich |
| Ultrasonic cell disruptor | | Ningbo Scientz |
| Scanning electron microscope | Nova Nano 450 | Thermo Fisher |
| Cryo-Transmission electron | Talos L120C | Thermo Fisher |
| microscope | | |
| Fluorescence microscope | T1 | Nikon |
| Blood Glucose Meter | Aviva | ACCU-CHEK, Roche |
| | | Pharmaceuticals, USA |
| ELISA Kits | Recombinant Human | Invitrogen |
| | Insulin ELISA Kits | |
| Continuous blood glucose | FreeStyle Libre H | Abbott |
| monitoring system | | |
| Insulin Glargine | Lantus | Sanofi |
| Protease inhibitors | MB2678 | MeilunBio |
| Phosphatase inhibitors | MB12707 | MeilunBio |
| RIPA lysis solution | P0013B | Beyotime |
| BCA Kits | P0012 | Beyotime |
| Laemmli Lysis buffer | 38733 | Sigma-Aldrich |
| PVDF membrane | 03010040001 | Sigma-Aldrich |
| Primary Antibody Dilution | P0023A | Beyotime |
| SuperSignal West Atto | A38554 | Thermo Fisher |
| Anti-Mouse TNFα | YT4689 | Immunoway |
| Anti-Mouse IL6 | DF6087 | Afinity |
| Anti-Mouse IL10 | DF6894 | Afinity |
| Anti-Mouse IL12A | AF5133 | Afinity |
| Anti-Mouse IL17A | DF6127 | Afinity |
| Anti-alpha smooth muscle Actin | ab240654 | Abcam |
| Anti-F4/80 | ab300421 | Abcam |
| Anti-rabbit IgG (Alexa Fluor ® | 4412 | Cell signaling |
| 488 Conjugate) | | |
| Anti-mouse IgG (Alexa Fluor ® | 8890 | Cell signaling |
| 594 Conjugate) | | |
| Anti-Phospho-Akt (Ser473) | 4060 | Cell signaling |
| Anti-Akt (pan) | 4691 | Cell signaling |
| Anti-β-Actin | db7283 | Diagbio |
| HRP AffiniPure Goat Anti-Rabbit | FDR007 | HANGZHOU FUDE |
| IgG (H + L) | | |

### Example 1

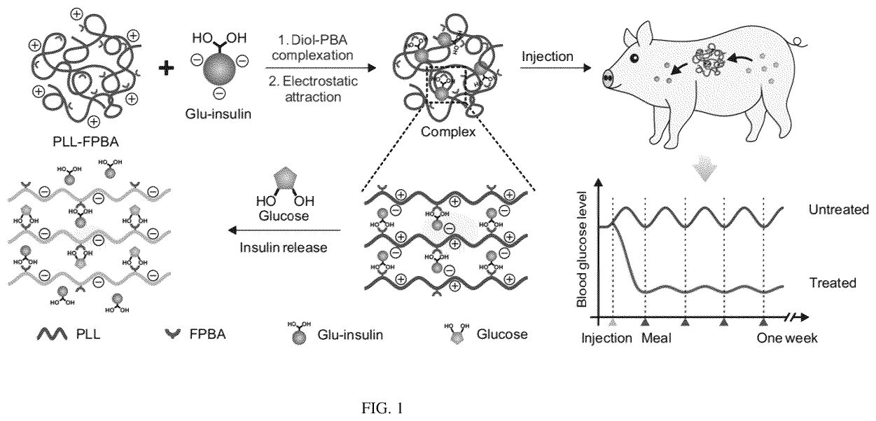

The preparation of the insulin complex and the glucose-responsive insulin release mechanism are shown in FIG. 1.

### 1. Preparation of Glu-Insulin

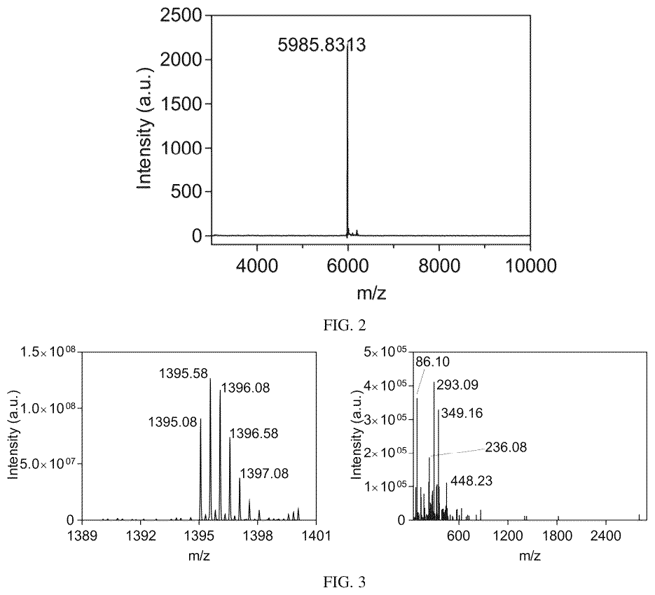

15.69 g of gluconic acid was dissolved in 40 mL of dimethyl sulfoxide (DMSO). 9 mmol of dicyclohexylcarbodiimide (DCC) and 10 mmol of N-hydroxysuccinimide (NHS) were dissolved in 5 mL of DMSO, and added to the gluconic acid solution. The mixture was stirred at room temperature overnight and the precipitate was filtered out. 0.8 g of insulin was dissolved in 10 mL of phosphate buffer (pH 7.4), and the filtrate was added to the insulin solution. The mixture was reacted in an ice bath for 2 hours. The reactant was dialyzed 3 times with 4 L deionized water or separated with an ion exchange column, and freeze-dried to obtain Glu-insulin. The MALDI-TOF mass spectrum of the prepared Glu-insulin is shown in FIG. 2. The results in FIG. 3 show that the modification site of gluconic acid on the recombinant human insulin is A1 (N-terminus of the α-chain).

### 2. Preparation of Poly-L-Lysine-4-Carboxy-3-Fluorophenylboronic Acid (PLL-FPBA)

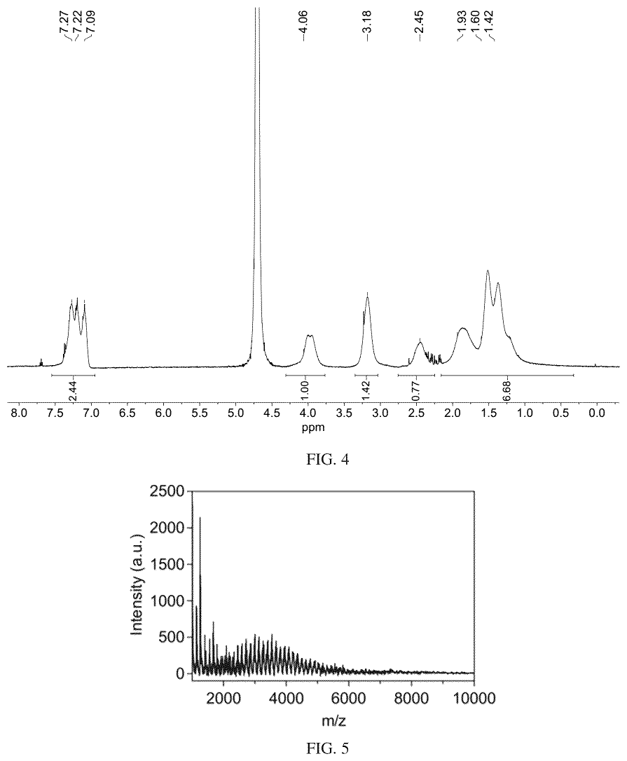

120 mg of FPBA-NHS was dissolved in 5 mL of DMSO. The mixture was added dropwise to 10 mL of phosphate buffered saline (PBS, 10 mM, pH 7.4) containing 100 mg of poly-L-lysine (PLL) (30 k-70 k). The pH was controlled at around 7. The FPBA-NHS solution was added and stirred for 30 min, and then dialyzed in deionized water (4 L). The resulting mixture was freeze-dried to obtain a white solid. The product was characterized by 1H NMR (refer to FIG. 4). The 1H NMR spectrum showed that 60% of the amino groups on PLL were modified with 4-carboxy-3-fluorophenylboronic acid (FPBA). PLL-FPBA was polydisperse (refer to FIG. 5). The presence of boron was confirmed by the 11B NMR spectrum (refer to FIG. 6).

### 3. Preparation of the Insulin Complex

1 mg of Glu-insulin and 1 mg of PLL-FPBA were dissolved in 0.1 mL of weakly acidic pure water (pH 3.0), respectively. 1 M NaOH aqueous solution was added. The pH was adjusted to 7.4 and white flocculent precipitate was formed. The white flocculent precipitate was collected by centrifugation, added to 1 mL of PBS, and stored in 4° C. (refrigerated).

### Example 2

In “3. Preparation of the insulin complex”, the amount of PLL-FPBA was changed to 2 mg. Other steps and conditions were the same as in Example 1.

### Example 3

In “3. Preparation of the insulin complex”, the amount of PLL-FPBA was changed to 1.5 mg. Other steps and conditions were the same as in Example 1.

### Example 4

In “3. Preparation of the insulin complex”, the amount of PLL-FPBA was changed to 0.5 mg. Other steps and conditions were the same as in Example 1.

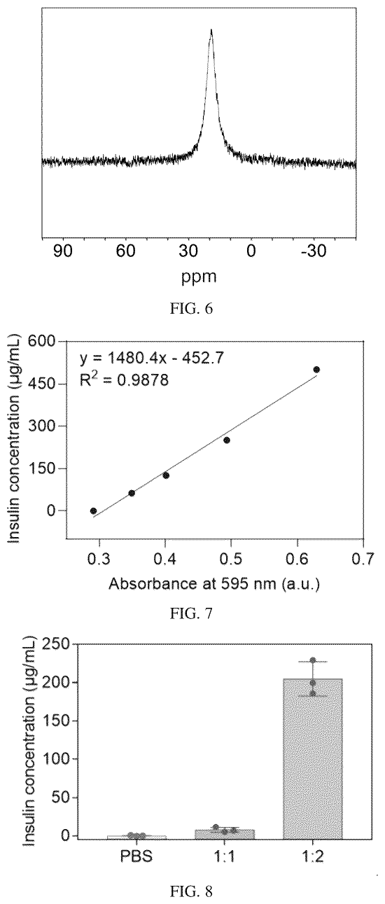

The free insulin in the supernatant was determined using Bradford reagent. Bradford reagent (200 μL) and 10 μL of the supernatant after centrifugation were added to a 96-well plate. The free insulin content in the supernatant was calculated based on its absorption value at 595 nm using the external standard method (refer to FIG. 7 for the standard diagram). The insulin encapsulation rate was calculated. According to the calculation, during the preparation of the insulin complexes of Examples 1 to 3, the encapsulation rates of insulin were all higher than 95%. As shown in FIG. 8, during the preparation of the insulin complex of Example 1, the encapsulation rate of insulin was higher than 95%. Whereas in Example 4, relatively more insulin was used, which exceeded the loading efficiency of the polymer. More free insulin was detected in the supernatant.

### Effect Example 1: In Vitro Activity Study of Glu-Insulin

The HepG2 cell line was obtained from the National Collection of Authenticated Cell Cultures (Shanghai, China). HepG2 cells were cultured in DMEM containing 10% fetal bovine serum and 1% penicillin/streptomycin. Cells were serum-starved for 12 h after the culture reached 80-90% confluence. Then, the cells were washed with PBS and added with medium containing recombinant human insulin or Glu-insulin for 10 min (0, 0.1, 1, 5, 10, 50, and 100 nM). The cells were washed with PBS and added ice-cold RIPA buffer (radioimmunoprecipitation assay buffer, Beyotime) containing protease and phosphatase inhibitors (MeilunBio) to conduct cell lysis. The lysate was centrifuged at 16,000 g for 10 min at 4° C. Protein concentration was determined by the BCA method (Beyotime), and samples were boiled in 1× Laemmli sample loading buffer (Bio-Rad) containing 2.5% β-mercaptoethanol. Proteins were separated by 10% SDS-PAGE gel and transferred onto a 0.45 μm PVDF membrane (Sigma-Aldrich). The membranes were blocked with 5% skim milk in PBST (phosphate buffered saline containing 0.1% Tween-20) and bound using antibodies [Anti-phospho-akt (CST, #4060, 1:2000), Anti-akt (CST, #4691, 1:1000), and Anti-β-Actin (Diagbio, #db7283, 1:1000)] in Primary Antibody Dilution (Beyotime). Horseradish peroxidase-conjugated secondary antibodies were used to bind primary antibodies according to the manufacturer's instructions. SuperSignal West Atto was used as chemiluminescent substrate (ECL, Thermo Fisher).

The results are shown in FIG. 9. In HepG2 cells, Glu-insulin showed activity similar to that of unmodified recombinant human insulin.

### Effect Example 2: Study on the Morphology of the Insulin Complex

### 2.1. Characterization of the Insulin Complex Morphology by Fluorescence Microscopy

0.7 mg of fluorescein isothiocyanate (FITC) was dissolved in 0.3 mL of DMSO. 10 mg of Glu-insulin (prepared according to step 1 of Example 1) was added to 5 mL of NaHCO3 (0.1 M). The mixture was stirred at room temperature overnight, dialyzed three times in 4 L of deionized water in the dark, and freeze-dried to obtain FITC-labeled Glu-insulin.

0.1 mg of water-soluble Cy5-NHS ester was dissolved in 0.02 mL of DMSO, 10 mg of PLL-FPBA (prepared according to step 2 of Example 1) was added to 5 mL of NaHCO3 (0.1 M). The mixture was stirred overnight in an ice bath, dialyzed three times in 4 L of deionized water in the dark, and freeze-dried to obtain Cy5-labeled PLL-FPBA.

The insulin complex was prepared according to step 3 of Example 1 using FITC-labeled Glu-insulin and Cy5-labeled PLL-FPBA.

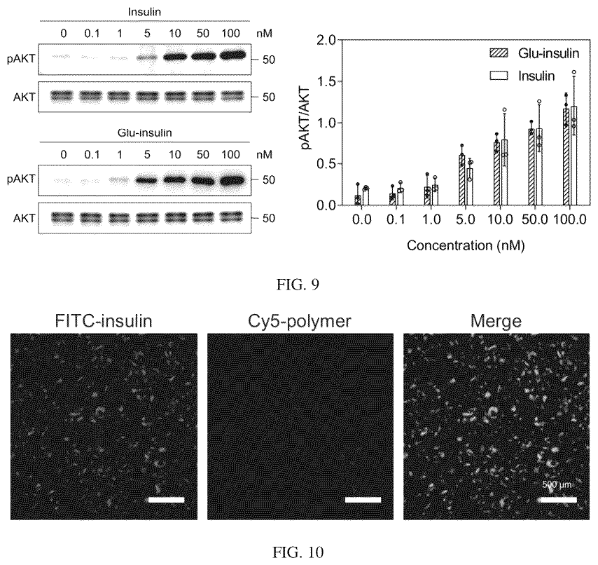

The results of observation using a fluorescence microscope (Model T1, Nikon) are shown in FIG. 10. The complexation of Glu-insulin and PLL-FPBA was further verified by the fluorescences of FITC-labeled Glu-insulin and Cy5-labeled PLL-FPBA overlap.

### 2.2 Characterization of the Insulin Complex Morphology by SEM and Cryo-TEM

The insulin complex obtained in Example 1 was added in water to make the insulin equivalent concentration 1 mg/mL. The complex was sonicated for 1 minute with an ultrasonic cell disruptor at 100 W power. The solution was dispersed in 1 mL water. After being diluted 10 times, it dropped onto copper grids. 10 μL of 5% uranyl acetate solution was added to the copper grids and stood for 10 min. The solution was removed with filter paper and the samples were observed using cryo-TEM. The same samples were placed on a silicon wafer, dried naturally, and observed by SEM. A porous and loose microstructure of the insulin complex was confirmed (refer to FIG. 11).

### Effect Example 3: In Vitro Glucose-Response Efficacy of the Insulin Complex

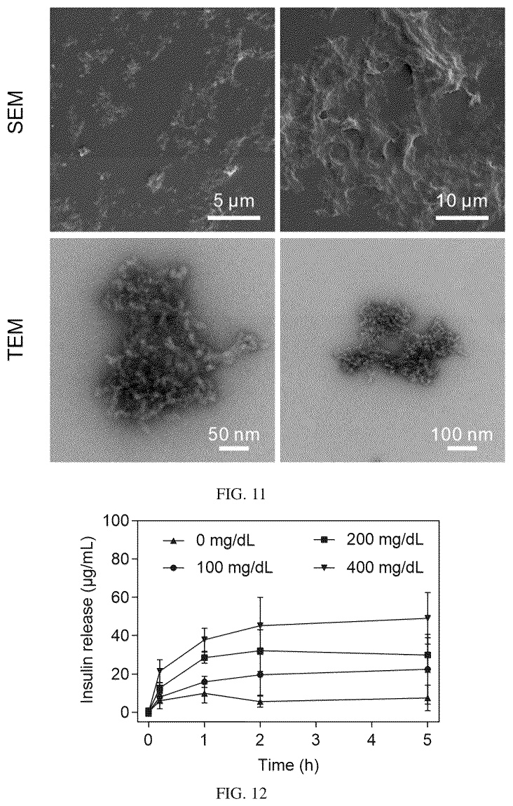

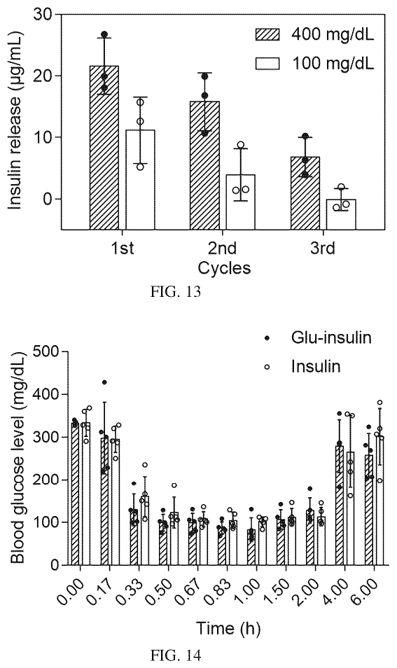

The glucose-responsive insulin release performance of the insulin complex prepared in Example 1 was evaluated in a PBS solution at pH 7.4 with four glucose concentrations of 0, 100, 200 and 400 mg/dL, respectively. In the absence of glucose, free Glu-insulin remained at a low level of around 10 μg/mL (refer to FIG. 12). When the glucose concentration increased to 100, 200 and 400 mg/dL, after 2 hours of incubation, the free Glu-insulin level increased to 19, 32 and 45 μg/mL, respectively. At this time, the free Glu-insulin level reached equilibrium and remained unchanged. After incubation in 400 mg/dL glucose solution for 0.2 h, the free Glu-insulin level reached 21 μg/mL, which was almost three times that in 100 mg/mL glucose solution (refer to FIG. 12). It was demonstrated that the insulin complex can release insulin in a glucose-dependent manner. The complex was exposed alternately to 100 and 400 mg/dL glucose solutions, and pulsatile insulin release was observed (refer to FIG. 13).

### Effect Example 4: Animal Efficacy Study

### 4.1 Animal Efficacy Study of Glu-Insulin

Type 1 diabetic mice (purchased from Hangzhou Medical College) were induced with a dose of 150 mg/kg streptozotocin. The diabetic mice were maintained on a standard diet and a circadian cycle of 12 h light, 12 h dark. Five mice each were subcutaneously injected with Glu-insulin and recombinant human insulin at an insulin equivalent dose of 1.5 mg/kg. The blood glucose was measured using a blood glucose meter. As shown in the results of FIG. 14, Glu-insulin still had a similar blood glucose-lowering effect as recombinant human insulin.

### 4.2 In Vivo Study of Glucose Control in Type 1 Diabetic Mice

A type 1 diabetic mouse model was established according to the above method, and type 1 diabetic mice with blood glucose higher than 300 mg/dL were selected for evaluation of the treatment effect. Each group (five mice) was subcutaneously injected with insulin glargine (50 U/kg) or insulin complex (20 mg/kg). The blood glucose was measured using a blood glucose meter.

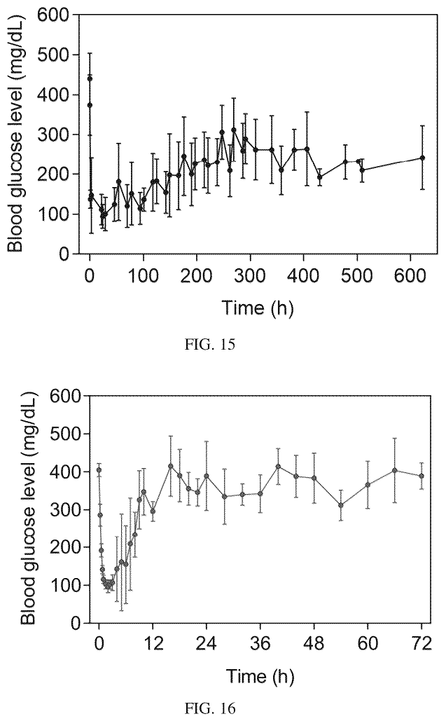

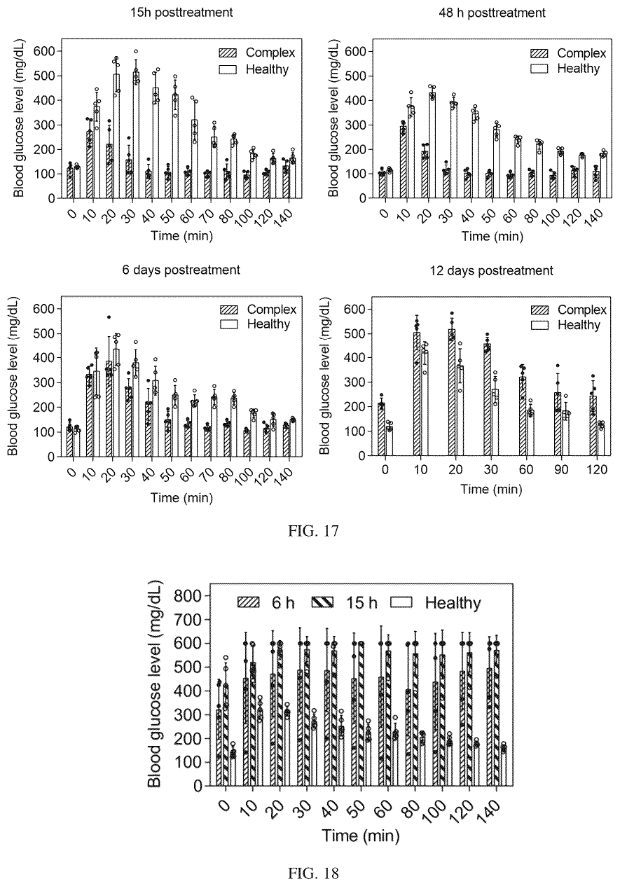

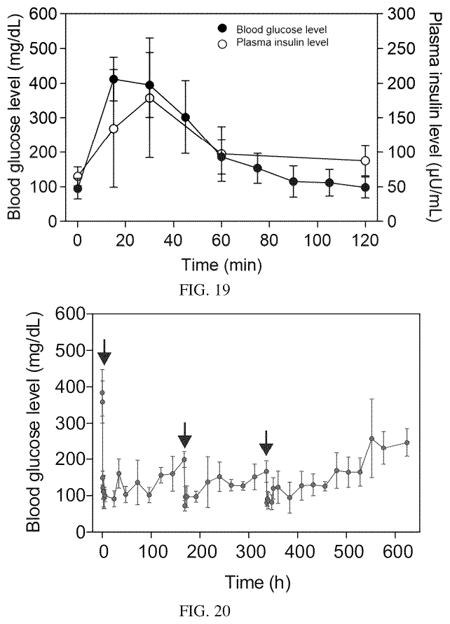

As shown in the results of FIG. 15, the blood glucose of mice was maintained below 200 mg/dL for more than one week, which was longer than the effective treatment time of the commercial long-acting insulin glargine (refer to FIG. 16) without severe hypoglycemia. The blood glucose regulation ability of the complex was further evaluated by intraperitoneal glucose tolerance test (IPGTT). After 15 hours, 48 hours, 6 days, and 12 days of insulin complex treatment, glucose solution was injected at 1.5 g/kg. As shown in the results of FIG. 17, mice treated with the complex could maintain stable blood glucose levels. As a control, diabetic mice treated with insulin glargine were unable to regulate blood glucose to normal levels at 6 and 15 hours after treatment (refer to FIG. 18). Insulin release stimulated by blood glucose was further investigated by IPGTT. Diabetic mice were pretreated with the complex (20 mg/kg) for 3 days and then intraperitoneally injected with glucose solution (3 g/kg). Plasma was collected, and blood glucose was measured. Plasma insulin levels were determined using an enzyme-linked immunosorbent assay (ELISA) kit (refer to FIG. 19). The insulin complex was injected every 168 hours (three injections at doses of 20 mg/kg, 14 mg/kg, and 14 mg/kg, respectively). The blood glucose could be controlled below 200 mg/dL for at least 22 days (refer to FIG. 20). The complex could achieve long-term blood glucose control in diabetic mice through a plurality of injections, and no obvious hypoglycemia was observed during this period.

### 4.3 In Vivo Study of Glucose Control in Type 1 Diabetic Minipigs

Six-month-old Bama minipigs were injected intravenously with streptozotocin (150 mg/kg) to induce type 1 diabetes. The minipigs were fed twice a day, and their blood glucose was managed daily with insulin glargine. It could be used for research after its blood glucose was stable for 1 month. The blood glucose was monitored using a continuous glucose monitoring system (FreeStyle Libre H, Abbott Laboratories, USA), and the blood glucose levels of the minipigs were all above 200 mg/dL. The complex treatment was given 48 hours after discontinuation of insulin glargine. The subcutaneous injection doses of insulin glargine in minipigs were 0.4, 0.5, and 0.6 U/kg, respectively. The subcutaneous injection dose of the complex for minipigs was 0.2 mg/kg, 0.3 mg/kg, and 0.3 mg/kg, respectively. As shown in the results of FIG. 21, a single injection of insulin glargine can control blood glucose level for no more than 24 hours and can reduce blood glucose to below the detection limit (less than 40 mg/dL). After injection of insulin glargine for seven consecutive days, blood glucose fluctuated between normoglycemia and hyperglycemia. Normal blood glucose could not be maintained for a long time, and the blood glucose levels were below 40 mg/dL. In contrast, all insulin complexes were able to control blood glucose to below 200 mg/mL, and hardly below 40 mg/dL. The complex was able to regulate blood glucose below 200 mg/dL in 2 minipigs for more than 120 hours and exerted a therapeutic effect for more than one week. Since minipigs are more clinically relevant than mice, the long-term blood glucose control ability and postprandial hyperglycemia regulation ability verified in the minipig model indicated that the complex was likely to regulate human blood glucose in the same way.

### Effect Example 5: Study on the Biocompatibility of the Complex

### 5.1 In Vivo Toxicity Assessment in Diabetic Mice

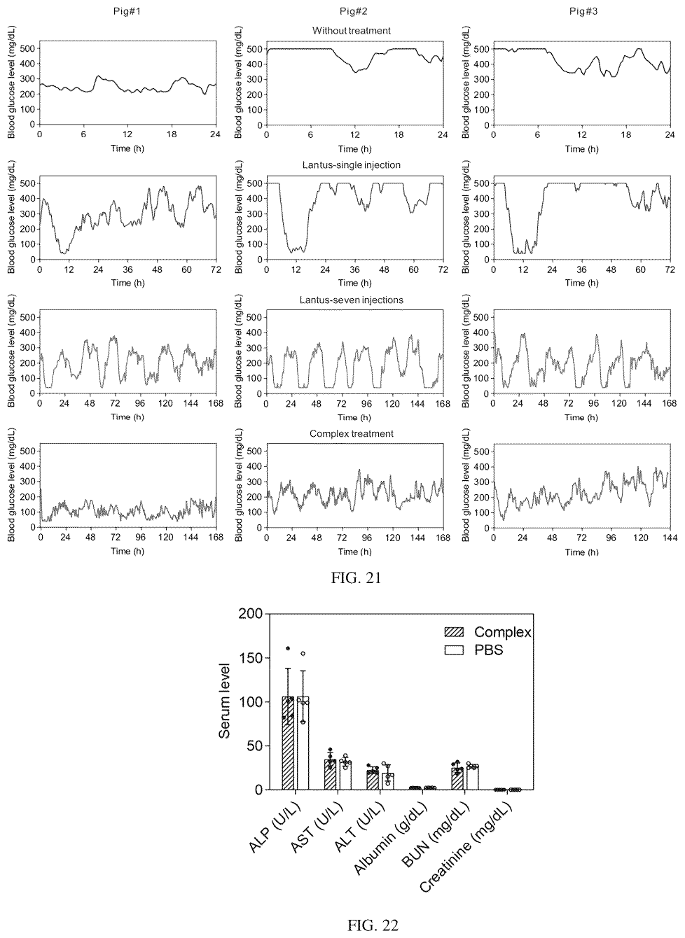

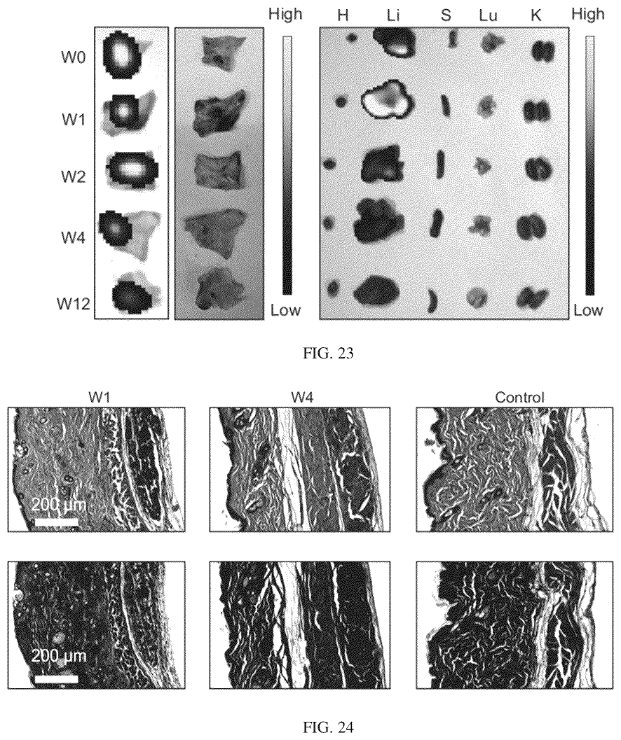

According to the above method, the type 1 diabetic mouse model was established, with 5 mice in each group. Each group was subcutaneously injected with PBS or the insulin complex (20 mg/kg). After one week, blood samples were collected and serum alkaline phosphatase (ALP), aspartate aminotransferase (AST), alanine aminotransferase (ALT), albumin (ALB), blood urea nitrogen (BUN) and creatinine levels were determined to evaluate the toxicity to liver and kidney (refer to FIG. 22). Compared with the mice in PBS injection group, the changes of various indicators in the complex group were not obvious, which indicated that PLL-FPBA had little toxicity to the liver and kidney. In addition, Cy5-labeled PLL-FPBA was injected subcutaneously, and the size of PLL-FPBA was visualized using an in vivo imaging system (IVIS Lumina III, PerkinElmer). As shown in the results of FIG. 23, the complex was mainly cleared by the liver. Hematoxylin-eosin (H&E) staining and Masson's trichrome (M&T) staining were used to evaluate the immune response of mice induced by the injected complex. Even four weeks after subcutaneous injection of the complex, no obvious neutrophil infiltration and obvious fibrous capsule were observed. (refer to FIG. 24).

### 5.2. Host Immune Response Evaluation of PLL-FPBA in Mice

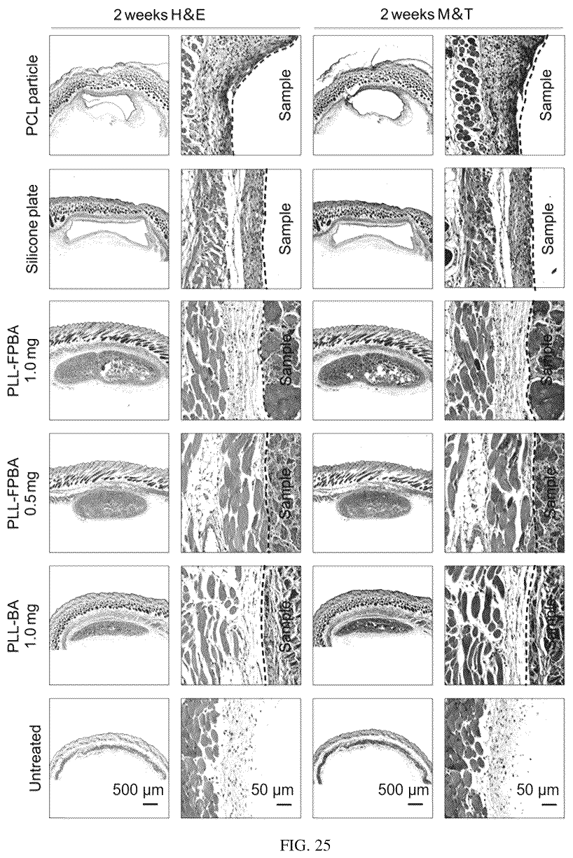

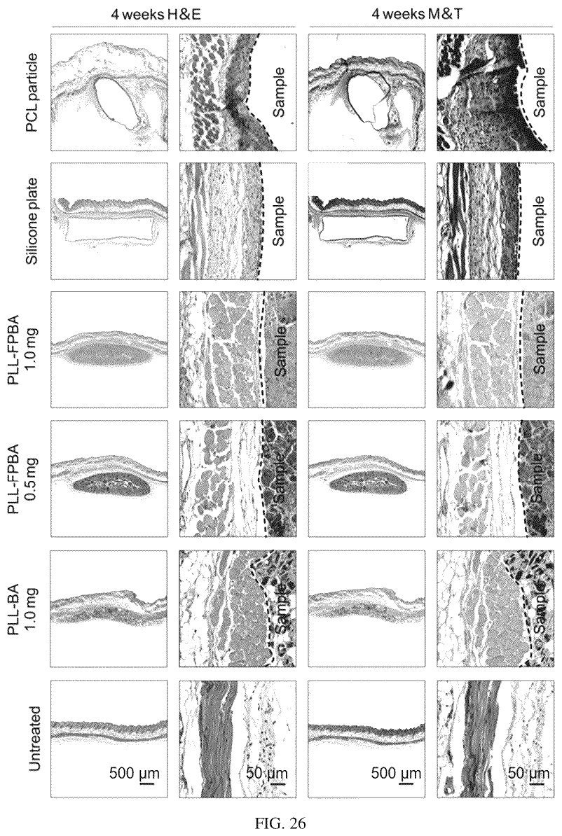

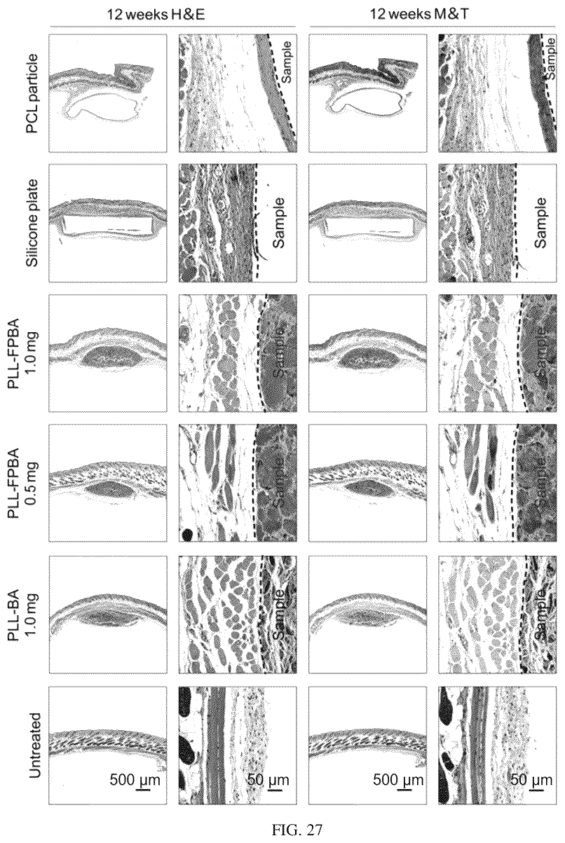

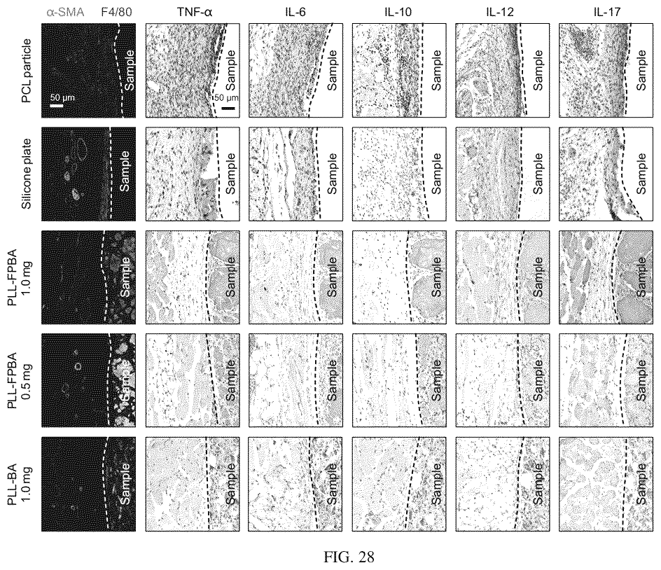

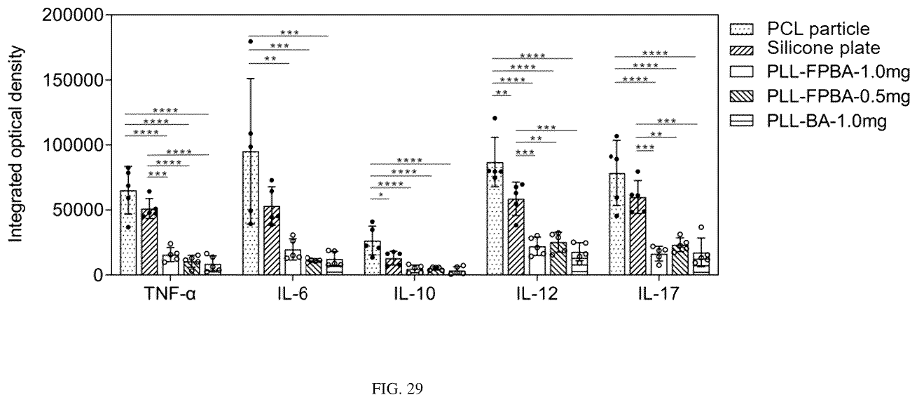

Six-week-old healthy C57BL/6 mice, five in each group, were implanted with PCL particles (diameter, 5 mm; average molecular weight 45,000, Sigma), silicone plates (diameter, 5 mm), PLL-FPBA (0.5 mg, 1.0 mg), and PLL-BA (1.0 mg, modified with 64% benzoic acid). All materials were sterilized. Animal surgery was performed under isoflurane anesthesia and aseptic conditions, and the skin was disinfected before and after surgery. A longitudinal incision of about 8 mm was made on the back of the mice for implantation of positive control, PCL particles and silicone plates. The incision was made far enough from the implantation site to avoid the impact of the wound. After the materials were implanted, the skin of mice was sutured. PLL-FPBA or PLL-BA was suspended in 0.1 mL of PBS and injected subcutaneously. The mice were euthanized on 2, 4, and 12 weeks after the implantation. The skin containing the implants was taken down and fixed with 4% paraformaldehyde for more than 24 hours, and embedded in paraffin. Untreated mice from the same batch served as blank controls. Each skin tissue was sliced with a thickness of 3 to 4 μm, and subjected to H&E and M&T staining. Immunofluorescence staining was performed for α-SMA and F4/80, and immunohistochemical staining was performed for cytokines TNF-α, IL-6, IL-10, IL-12, and IL-17. Immunofluorescence images were recorded on a laser scanning confocal microscope (ECLIPSE Ti2, Nikon). H&E, M&T, and immunohistochemical images were acquired using a digital slide scanner (VS200, Olympus). The positive staining analysis of immunohistochemical images was performed on ImageJ2 (Fiji) (within 100 μm from the implant interface). As shown in FIG. 25, 2 weeks after implantation, the implants had similar sizes under the skin. No obvious fibrous capsule was found around PLL-FPBA at 2, 4, and 12 weeks after subcutaneous injection (refer to FIGS. 25-27). In contrast, PCL particles and silicone plates triggered a thick, dense collagen or fiber layer around the implants. On 2 and 4 weeks after the implantation, a thicker layer of immune cells was on the surface of PCL particles and silicone plates. In contrast, the number of immune cells surrounding PLL-FPBA and PLL-BA implants were negligible. The macrophage marker F4/80 was labeled in red by immunofluorescence staining. A lower red fluorescence density was observed around the PLL-FPBA implants compared with that around the PCL particles and silicone plates. Cytokine levels around the implants were also studied (refer to FIGS. 28-29). The accumulation of TNF-α, IL-6, IL-10, IL-12, and IL-17 was the most serious around the PCL particle implants. The silicone plates also caused significant cytokine accumulation. In contrast, low levels of all cytokines were identified in the space surrounding both PLL-FPBA and PLL-BA, which indicated that the minor role of FPBA in reducing host immune response. Of note, the size of the implants of PLL-FPBA and PLL-BA decreased gradually over time due to the inherent biodegradability of the PLL backbone (refer to FIGS. 25-27). The slow removal of PLL-FPBA resulted in weak adhesion of collagen fibers on the implant surface, which prevented the formation of fibrous capsule. In addition, α-SMA was observed in the silicone plates group and PLL-FPBA group, which had the potential to stimulate angiogenesis.

The insulin complex developed in the present disclosure demonstrates sustained insulin release, as confirmed by in vitro and in vivo glucose-response tests, as well as pharmacodynamic evaluations in mouse and minipig models. These results indicate that the complex enables slow and prolonged insulin release, allowing for higher single-dose administration and reduced injection frequency. Additionally, the PLL-FPBA component exhibits low immunogenicity, minimizing host immune responses and preventing subcutaneous fibrous capsule formation, thereby supporting long-term insulin delivery.

1. A glucose-responsive insulin complex, comprising phenylboronic acid-based polylysine and insulin with a diol structure, wherein interaction forces comprise: (1) a dynamic electrostatic attraction, and (2) a diol-phenylboronic acid (diol-PBA) complexation force between the phenylboronic acid-based polylysine and the insulin with the diol structure.

2. The glucose-responsive insulin complex according to claim 1, wherein in the phenylboronic acid-based polylysine, a phenylboronic acid group is grafted onto a side chain of a polylysine via an amide bond;
wherein a phenylboronic acid grafting rate of the phenylboronic acid-based polylysine is 25% to 75%.

3. The glucose-responsive insulin complex according to claim 1, wherein polylysine in the phenylboronic acid-based polylysine is at least one of an E-polylysine or an L-polylysine;
wherein a molecular weight of the polylysine is 2 k to 1000 k.

4. The glucose-responsive insulin complex according to claim 1, wherein a phenylboronic acid group in the phenylboronic acid-based polylysine is a phenylboronic acid group substituted or unsubstituted with a substituent.

5. The glucose-responsive insulin complex according to claim 1, wherein the insulin with the diol structure is prepared from insulin and a compound containing ortho- or meta-hydroxyl groups;
wherein an insulin loading rate in the glucose-responsive insulin complex is 5% to 80%, the percentage is a mass percentage of the insulin with the diol structure in the glucose-responsive insulin complex to the glucose-responsive insulin complex.

6. A method for preparing the glucose-responsive insulin complex according to claim 1, comprising following steps:
mixing a solution of the phenylboronic acid-based polylysine with a solution of the insulin with the diol structure; and adjusting pH to a range of 6.5 to 8.0.

7. The method for preparing the glucose-responsive insulin complex according to claim 6, wherein a mass ratio of the insulin with the diol structure to the phenylboronic acid-based polylysine is 1:(0.5-10);
wherein a concentration of the solution of the phenylboronic acid-based polylysine is 1 to 200 mg/mL;
wherein a preparation method of the solution of the phenylboronic acid-based polylysine comprises following steps:
dissolving the phenylboronic acid-based polylysine in weakly acidic water;
wherein a pH of the weakly acidic water is in a range of 2.0 to 7.0.

8. The method for preparing the glucose-responsive insulin complex according to claim 6, wherein a concentration of the solution of the insulin with the diol structure is 1 to 200 mg/mL;
wherein a preparation method of the aqueous-solution of the insulin with the diol structure comprises following steps:
dissolving the insulin with the diol structure in weakly acidic water;
wherein a pH of the weakly acidic water is 2.0 to 7.0.

9. The method for preparing the glucose-responsive insulin complex according to claim 6, wherein adjusting the pH to the range of 6.5 to 8.0 comprises adjusting the pH to a range of 6.5 to 7.4;
wherein a step of centrifugation is further comprised after adjusting the pH;
wherein an insulin encapsulation rate of the method is above 90%.

10. A use of the glucose-responsive insulin complex according to claim 1 in preparing a drug for treating diabetes.

11. A method for treating diabetes in a patient, comprising administering to the patient the glucose-responsive insulin complex according to claim 1.

12. A drug for treating diabetic patients, comprising the glucose-responsive insulin complex according to claim 1.

13. A glucose-responsive insulin complex according to claim 1 for use in treating diabetes.

14. The glucose-responsive insulin complex according to claim 3, wherein the polylysine in the phenylboronic acid-based polylysine is a phenylboronic acid-based L-polylysine.

15. The glucose-responsive insulin complex according to claim 3, wherein the molecular weight of the polylysine is 30 k to 70 k.

16. The glucose-responsive insulin complex according to claim 4, wherein the substituent is one or more of a halogen and a nitro group.

17. The glucose-responsive insulin complex according to claim 4, wherein the phenylboronic acid group is selected from one or more of following groups:

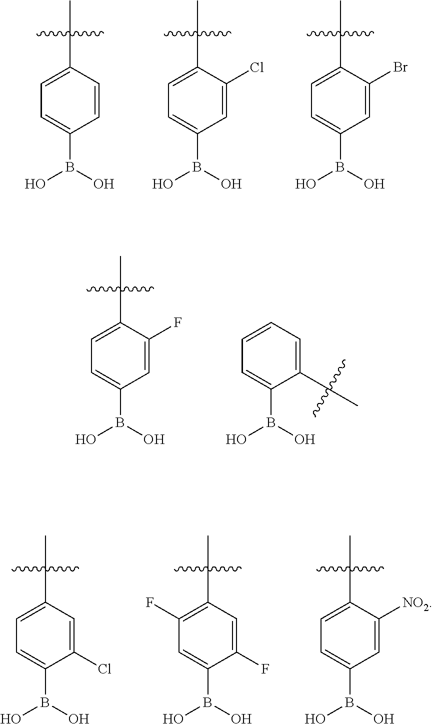

18. The glucose-responsive insulin complex according to claim 5, wherein the compound containing ortho- or meta-hydroxyl groups is one or more of gluconic acid, dopamine, fructose, lactose, ribose, deoxyribose, β-D-pyranoside, trehalose, or maltose.

19. The glucose-responsive insulin complex according to claim 5, wherein the compound containing ortho- or meta-hydroxyl groups is gluconic acid.

20. The method for preparing the glucose-responsive insulin complex according to claim 7, wherein the weakly acidic water is phosphate buffer, deionized water or pure water.
